(19) United States
(12) Patent Application Publication
(10) Pub. No.: US 2012/0150033 A1
(43) Pub. Date: Jun. 14, 2012

(54) ANTIMICROBIAL ULTRASOUND TRANSMISSION GEL

(76) Inventor: LOREN CURTIS RAUCH, Oakland, CA (US)

(21) Appl. No.: 13/179,249

(22) Filed: Jul. 8, 2011

Related U.S. Application Data
(60) Provisional application No. 61/422,529, filed on Dec. 13, 2010.

(51) Int. Cl. A61B 8/00; A61K 31/155

(52) U.S. Cl. 600/437; 514/635

(57) ABSTRACT

An antimicrobial ultrasound transmitting gel has an ultrasound-transmitting gel and an effective amount of one or more antimicrobial additives. The gel may further include one or more skin cleansers to provide a gel that may be used to both decontaminate the skin and for the performance of the ultrasound.

### CROSS-REFERENCE TO RELATED APPLICATIONS

This application claims the benefit of priority of U.S. provisional patent application No. 61/422,529, filed Dec. 13, 2010, which is herein incorporated by reference.

### BACKGROUND OF THE INVENTION

The present invention relates to ultrasound gels and, more particularly, to an antimicrobial ultrasound transmission gel that may reduce infection and/or simplify ultrasound guidance of invasive procedures.

Ultrasound is increasingly used and is becoming standard to facilitate many invasive procedures such as aspiration, biopsy, tube or vascular (peripheral or central) line placement. Use of ultrasound guidance during invasive medical procedures can aid in identifying fluid collections and anatomic or vascular structures and can aid in guiding a catheter, drain, probe or other device into the best location while minimizing complications such as procedure failure or damage to vascular or other structures. Previously, the method used for these types of invasive procedures was the “landmark technique” or “blind.” This real-time ultrasound guidance can decrease complications, increase success rates, and improve patient care.

Ultrasound usually requires an ultrasound transmitting gel, applied to a patient's skin or bodily surface, to facilitate performance. Ultrasound gel is typically water-based and contains humectants (water-absorbing and retaining substances) such as glycerin and glycols such as propylene glycol.

Typically, preservatives are included in the gel to prevent microbial degradation of the gel and to prevent the growth of pathogenic microorganisms. Examples include: phenoxyethanol, methyl chloride isothiazolynone, imidazolinyl urea, and EDTA. These compounds are formulated only to prevent bacterial growth in the gel. They do not reliably disinfect or kill microbes on skin surfaces.

Alcohol gels also may have inherent antimicrobial properties yet have not been used for ultrasound because, first, they may damage the expensive ultrasound transducer probe. Second, they are irritating to non-intact skin and mucosal surfaces.

When performing invasive medical procedures, contamination and infection may occur. Infections related to health care devices and other health care-related infections are an increasing concern in health care delivery. The U.S. Department of Health and Human Services has been calling for the reduction of health-care-associated infections, noting in particular that central line-associated blood stream infections are an important type with high mortality. These types of infections can be due to either inadequate, cleaning of the skin or body surface prior to the procedure or to contamination during or after the procedure itself.

To reduce risk of infection, performance of invasive medical procedures requires sterile technique, which involves preparatory skin cleansing and decontamination before the procedure, usually with an antimicrobial skin cleanser of sufficient strength, typically containing povidone-iodine, chlorhexidine or a similar antimicrobial substance in tincture or solution. Also, sterile technique typically includes hand cleansing; using sterile gloves, drapes and often other garments or covers; and using sterile devices and barriers to maintain sterility during and after the procedure. Despite these precautions, initial decontamination may be inadequate and/or secondary contamination can occur, both of which can cause infection.

Use of ultrasound guidance during invasive procedures can increase risk of inadequate decontamination and/or secondary contamination. Prevention of ultrasound-related contamination has conventionally required use of time-consuming and exacting sterile ultrasound preparations and technique.

To do an invasive procedure with ultrasound guidance requires clean ultrasound equipment and sterile gel and probe covers. Usually an assistant is also needed to help to hold the ultrasound probe and maintain sterility while applying gel inside the probe cover, which is placed over the transducer. Next the sterile ultrasound-transmitting gel is placed over the body surface where the skin preparation step was completed and often additional gel is applied outside the sterile probe cover.

The current need to both cleanse the skin and apply an additional ultrasound gel that does not contain significant antimicrobial properties increases complexity. Complexity may also increase risk of contamination due to greater chance of error in the sterile procedure. Additionally, ultrasound gel that lacks significant antimicrobial properties may contribute to inadequate sterilization, contamination and infection. First, the preparatory antimicrobial tincture or solution can be wiped off or diluted by application of an ultrasound gel that is applied during the time the tincture or solution needs to decontaminate the skin. Second, the application of ultrasound gel may decrease the post-procedure protective effect that is delivered by the residual antimicrobial substance(s) left on the skin from the tincture or solution.

As can be seen, there is a need for an ultrasound gel having at least one significant antimicrobial additive. An additional option is an ultrasound gel having at least one significant antimicrobial additive with additional skin cleanser to make a single skin cleansing, decontamination and ultrasound transmission gel product.

### SUMMARY OF THE INVENTION

In one aspect of the present invention, an antimicrobial ultrasound transmitting gel comprises a gel adapted to permit transmission of ultrasound waves; and at least one antimicrobial compound mixed with the gel.

In another aspect of the present invention, an antimicrobial ultrasound transmitting gel comprises a gel adapted to permit transmission of ultrasound waves; at least one antimicrobial compound mixed with the gel; and at least one skin cleanser.

In a further aspect of the present invention, a method for performing invasive ultrasound-facilitated medical procedures comprises decontaminating a patient's skin at a site of the medical procedure; and applying an antimicrobial ultrasound transmitting gel to the patient's skin at the site of the medical procedure, the antimicrobial ultrasound transmitting gel comprising a gel adapted to permit transmission of ultrasound waves and at least one antimicrobial compound mixed with the gel.

These and other features, aspects and advantages of the present invention will become better understood with reference to the following drawings, description and claims.

### BRIEF DESCRIPTION OF THE DRAWINGS

FIG. 1 is an elevation view of an ultrasound gel in use according to an exemplary embodiment of the present invention;

FIG. 2 is a perspective view of the ultrasound gel of FIG. 1, in use in an invasive medical procedure; and

FIG. 3 is another perspective view of the ultrasound gel of FIG. 1 in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Various inventive features are described below that can each be used independently of one another or in combination with other features.

Broadly, an embodiment of the present invention provides a composition having an ultrasound-transmitting gel and an effective amount of an antimicrobial additive. The gel may further include one or more skin cleansers to provide a gel that may be used to both decontaminate the skin and for the performance of the ultrasound.

For the purpose of this disclosure, antiseptic or antimicrobial substances are substances that kill or slow the growth of infective microorganisms. Aseptic describes the absence of infectious microorganisms and an aseptic technique describes techniques performed during procedures that maintain sterility by using sterile or clean instruments, surfaces and covers such as gloves. A cleanser is a substance such as soap that facilitates the removal of dirt and other debris. An effective amount of an antimicrobial additive may refer to an amount of antimicrobial additive that will reduce infection by killing or inhibiting infective organisms, such as bacteria.

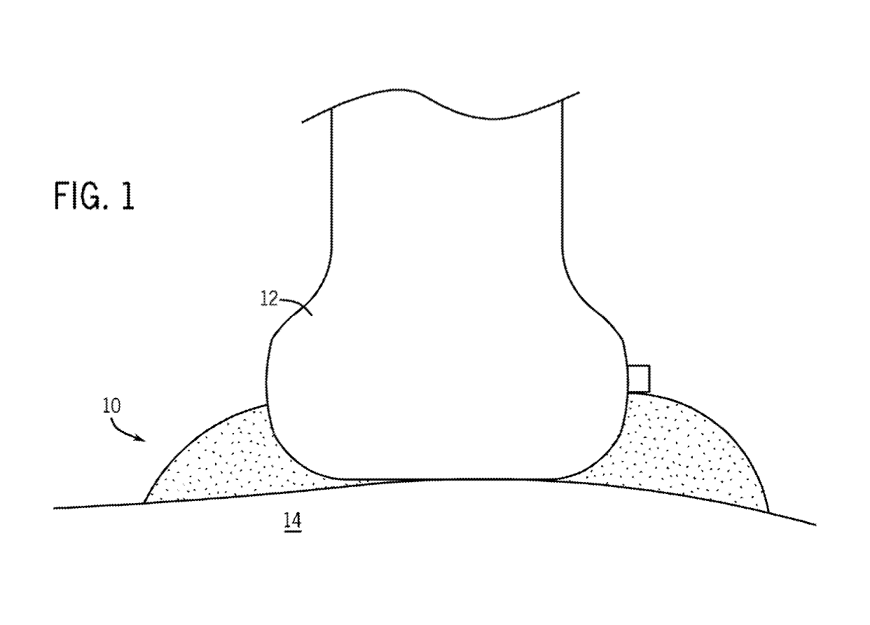

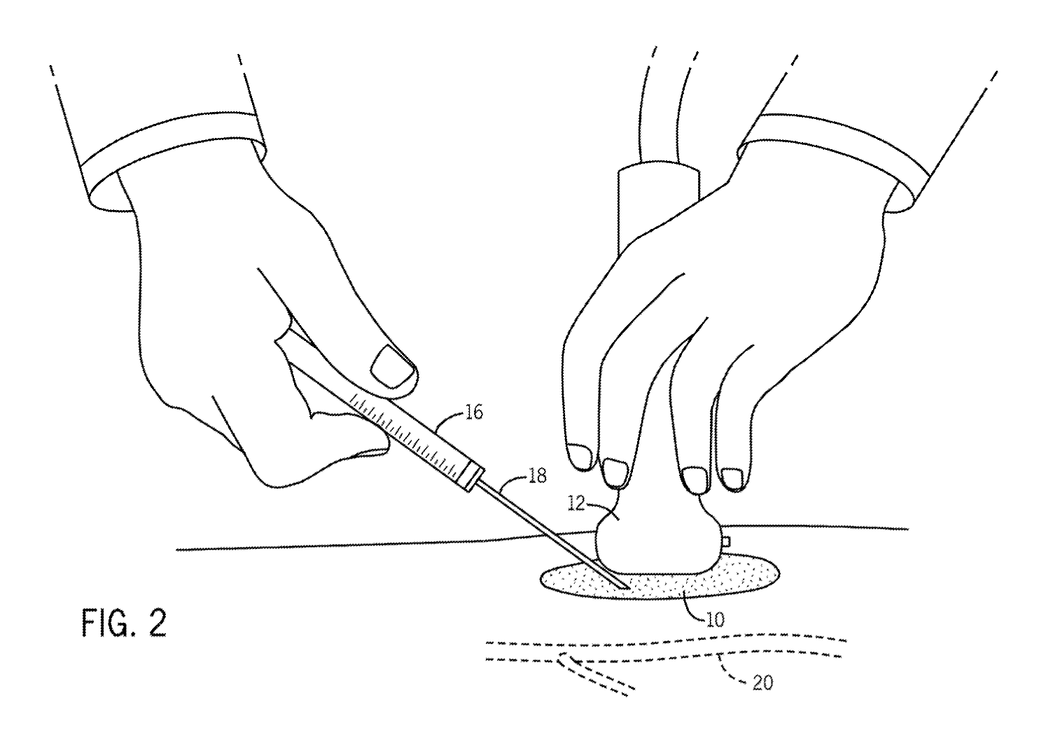

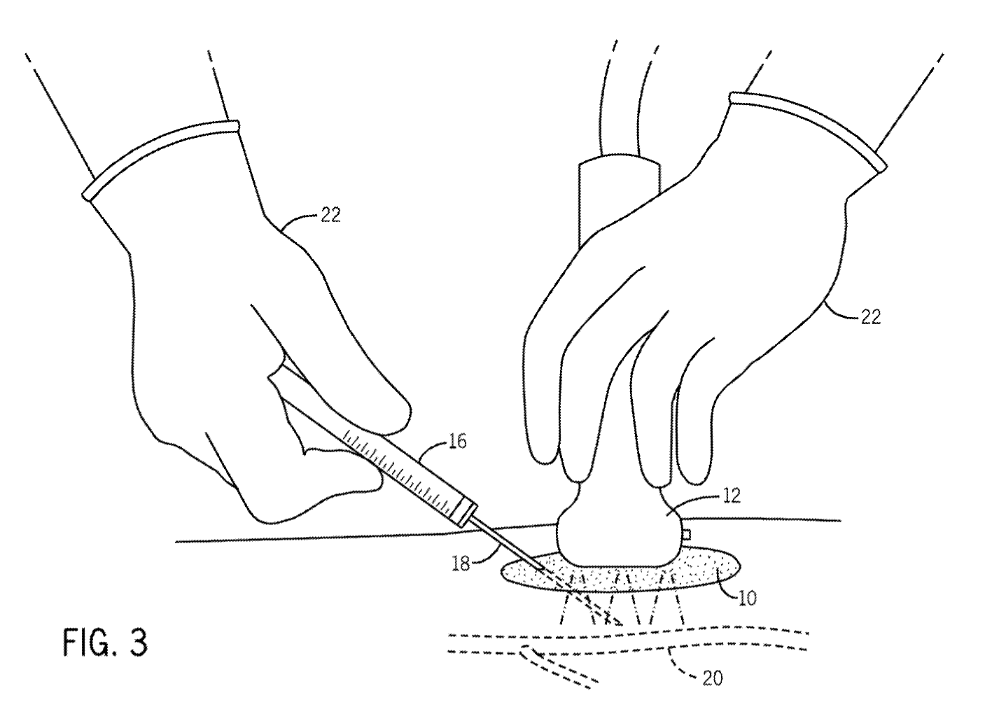

Referring to FIGS. 1 through 3, an ultrasound transmission gel 10 may be used to facilitate the transmission of ultrasound signals (such as ultrasound sound waves) from an ultrasound probe 12 into a body surface 14. Because the ultrasound waves travel poorly through air and the body surface will not exactly conform to the surface of the probe 12, there must be a medium to carry the energy from the probe 12 to the skin. When a probe cover (not shown) is used, additional gel may be applied inside the cover to improve ultrasound transmission. The gel used inside the cover may be antimicrobial gel 10, according to the present invention, or may be standard ultrasound gel.

The ultrasound gel 10 may be used in an invasive medical procedure. For example, when the specific placement of a needle 18, attached to a syringe 16, relative to a blood vessel 20, is needed, ultrasound may be used to facilitate the placement of the needle 18 inside the body 14. The ultrasound gel 10 may provide both ultrasound transmission properties as well as antimicrobial properties. In some embodiments, the ultrasound gel 10 may further include cleansing properties, as discussed in greater detail below.

The ultrasound gel 10 may reduce infection by killing or inhibiting infective organisms, such as bacteria, for example, on the body surface being incised or punctured during a medical procedure, or on potentially contaminated gloved hands 22, or on inadequately cleaned devices. The antimicrobial agent may be, for example, chlorhexidine gluconate, at a concentration from about 1% to about 4% by weight. Alternatively, providine-iodine can be used is concentrations between about 2.5% to about 10%. Alternatively, the antimicrobial agent Triclosan® at a concentration between about 0.1% and about 4% can be used. Including antimicrobials at these concentrations would provide antimicrobial properties to the ultrasound gel and can be expected to result in therapeutic residual antimicrobial concentrations on the skin after use.

The ultrasound gel 10 may include one or more skin cleansers or surfactants as part of the gel composition. The cleansers may aid in removing surface material while not necessarily having inherent antimicrobial properties. The skin cleanser may be, for example, sodium lauryl sulfate at a concentration of about 5% to about 20%.

The ultrasound gel 10 may be formed by homogeneously combining a conventional ultrasound transmission gel with at least one antimicrobial compound. This combination may be done in such a way as to maintain the ultrasound transmitting qualities as well as antimicrobial qualities. The gel 10 may also contain at least one skin-cleansing agent homogeneously combined therein. Similarly, the combination may be done in such a way as to maintain the ultrasound transmitting qualities.

To use the gel 10, a practitioner may first either use the ultrasound gel of the present invention as a skin decontamination and cleansing preparation for the medical procedure or apply the gel for the ultrasound procedure after another method of skin decontamination is used. The gel 10 may be reapplied as necessary to aid in the performance of the ultrasound. The gel 10 may also be used primarily for skin decontamination and cleansing. The gel 10 may also be used primarily as an ultrasound transmitting gel. The gel 10 may be used on body surfaces other than skin.

It should be understood, of course, that the foregoing relates to exemplary embodiments of the invention and that modifications may be made without departing from the spirit and scope of the invention as set forth in the following claims.

What is claimed is:

1. An antimicrobial ultrasound transmitting gel comprising:
a gel adapted to permit transmission of ultrasound waves; and
at least one antimicrobial compound mixed with the gel.

2. The antimicrobial ultrasound transmitting gel of claim 1, further comprising at least one skin cleanser.

3. The antimicrobial ultrasound transmitting gel of claim 1, wherein the antimicrobial compound is present in an amount effective to kill or inhibit infective organisms.

4. The antimicrobial ultrasound transmitting gel of claim 1, wherein the antimicrobial compound is homogeneously mixed with the gel and the ultrasound transmitting qualities of the gel are maintained.

5. The antimicrobial ultrasound transmitting gel of claim 2, wherein the skin cleanser is homogeneously mixed with the gel and the ultrasound transmitting qualities of the gel are maintained.

6. An antimicrobial ultrasound transmitting gel comprising:
a gel adapted to permit transmission of ultrasound waves;
at least one antimicrobial compound mixed with the gel; and
at least one skin cleanser.

7. The antimicrobial ultrasound transmitting gel of claim 6, wherein:
the antimicrobial compound is present in an amount effective to kill or inhibit infective organisms;
the antimicrobial compound is homogeneously mixed with the gel and the ultrasound transmitting qualities of the gel are maintained; and
the skin cleanser is homogeneously mixed with the gel and the ultrasound transmitting qualities of the gel are maintained.

8. A method for performing invasive ultrasound facilitated medical procedures, comprising:
decontaminating a patient's skin at a site of the medical procedure; and
applying an antimicrobial ultrasound transmitting gel to the patient's skin at the site of the medical procedure, the antimicrobial ultrasound transmitting gel comprising a gel adapted to permit transmission of ultrasound waves and at least one antimicrobial compound mixed with the gel.

9. The method of claim 8, wherein the antimicrobial ultrasound transmitting gel includes at least one skin cleanser.

10. The method of claim 9, wherein the step for decontaminating the patient's skin is performed with the antimicrobial ultrasound transmitting gel.
